(19) United States
(12) Patent Application Publication
(10) Pub. No.: US 2009/0222061 A1
(43) Pub. Date: Sep. 3, 2009

(54) PYRODE NEUROSTIMULATOR

(76) Inventor: Gordon W. Culp, Van Nuys, CA (US)

Correspondence Address: Steven E. Kahm, 3140 Chowen Ave. S. #328, Minneapolis, MN, 55416, US

(21) Appl. No.: 11/419,581

(22) Filed: May 22, 2006

(51) Int. Cl. A61F 9/007; A61N 1/04

(52) U.S. Cl. 607/54

(57) ABSTRACT

A pyrode converts near-infrared light to an electric field that wirelessly stimulates nerve tissue at one or more locations in a host body, such as in a retina, a cochlea, a heart axon, or a motor nerve. Partial vision is restored when a pyrode implanted in a retina is activated by a scanner. Only the pyrode is implanted in the retina, whereas all other components remain external to the host body. Nerves other than the retina are stimulated by a pyrode coupled to a source of photonic energy by an optical conduit. In one preferred embodiment the photonic energy source is implanted in the host body. In another preferred embodiment, photonic energy from an external source is piped to the pyrode by way of an optical conduit via a percutaneous lead. Electrooptic diverters enable sequential activation of multiple pyrodes branched from a main conduit.

### BACKGROUND OF THE INVENTION

1. Field of the Invention

This invention relates to photonic apparatus implanted in the body for stimulation of nerve tissue.

2. Description of Related Art

“Pyrode,” a term coined for the present invention, is an intended concatenation of “pyro” (fire) and “electrode.” Pyrode is defined herein as a body of solid, electrically non-conducting matter having a surface onto which an external source impinges a pulse of photonic energy (sudden feeble warming) that is converted internal to said body into an intense electric field manifested external to and proximate said surface.

Functional neural tissue, while remaining responsive to stimulation, is known to become disconnected from the nervous system, but may be reactivated when properly stimulated, for example by a pulsed electric field. Stimulators of the prior art rely on an electrical conductor to carry pulses from an electric generator to the stimulation site, and on a metallic electrode to deliver electric charge to the tissue. Prior art metallic stimulators having multiple stimulation electrodes become very complicated when a large number of electrodes is desired. Delivery electrode arrays of the prior art must be connected to a generator module by a multi-conductor cable. Furthermore, prior art stimulators require at least one energy storage and control module implanted nearby, which significantly increases the invasiveness of the implantation procedure. The conductors of known metallic stimulators that lie near an electrode interfere with the shape of the stimulating electric field of that electrode. Metals also entail problems with fatigue, biocompatibility, electrolytic polarization, and corrosion. An improved neurostimulating means would not use metals for stimulation electrodes, and would not require multi-conductor cables for interconnections. An improved neurostimulator for the eye would require implanting only a single non-metallic element therein, and would relegate all other components to locations outside the body in order to minimize the invasiveness of implantation surgery.

The spatial resolution of prior art retinal stimulators is undesirably coarse because it is difficult to fabricate an array comprising a plethora of closely spaced metallic electrodes. Known metallic stimulating arrays have electrodes in fixed positions, precluding any possibility of locating a stimulus in the exact desired optimum position. An improved stimulator would allow a stimulus to be applied anywhere in the sensitive area, thereby exploiting highly effective regions selectively. Furthermore, an improved stimulator would enable control of the size of a stimulated area, and would provide tracking of a stimulation zone when relative motion occurs between a controlling means and a stimulating means.

Protecting prior art metallic stimulating electrodes with electrical insulation increases the distance between an electrode and the nerve tissue to be stimulated, thereby increasing the power of stimulation needed to affect a desired level of activation, which in turn reduces the reliability and longevity of the stimulation process. Covering prior art metallic multi-conductor cables with electrical insulation increases the bulk of the cable, increases the invasiveness of the implantation surgery, and increases the risk of tissue dysfunction due to increased-thickness long-term encapsulation. An improved stimulation means would minimize problems traceable to electrical insulation by eliminating same.

Known stimulation means comprising multiple metallic spikes intended to pierce nerve tissue require an electrically insulated multi-conductor cable to selectively connect a switchable electric power source to each spike. Quiescent spikes surrounding an activated spike short circuit the preponderance of electric field from the activated spike. What little field remains is highly distorted and may not contribute significantly to the stimulation of a nerve. Further, very fine metal spike-like electrodes are prone to faster electrolytic erosion at the tip because the tip geometry produces the highest local field intensity. An improved stimulator may consist of one or more electrically non-conducting spikes, wherein spikes surrounding an activated spike tend to concentrate the stimulating electric field.

Applicant's U.S. Pat. Nos. 5,281,899; 5,043,621 and 4,928,030 teach a ceramic body divided into at least three mechanically additive, but independently electrically addressable, portions, two portions for positioning in directions within a plane, and one portion for positioning in a direction normal to the plane. An improved scanner may place a steering lens in the plane to provide a combination of focus spot positioning, dynamic focus, and control of focal spot size of a light beam. An improved scanner actuator would control beam motion at video rates, and would excel at positioning precision, for example, as demonstrated when used in the well known scanning tunneling microscope. Positioning may be non-sinusoidal. Other positioners, inexhaustibly including electromagnetic, electrostatic, electrophoretic, and magnetooptic actuators lie within the inventive scope of an improved nerve stimulation apparatus. Also falling within the scope of an improved apparatus is a prior art light source consisting of an array of light emitters that is activated in concert with an a deflection means having beam steering, or array positioning, capability only sufficient to make “trim” adjustments commensurate with the inter-pixel spacing in the light emitting array, in order to provide what is tantamount to unlimited areal addressability.

A pyrode body may be composed of pyroelectric material, such as poly(vinylidene difluoride) (PVDF), wherein lattice pairs of positive and negative ions are closely spaced. Responsive to a sudden feeble warming, thermal excitation changes the distance between the charge pairs, which in turn creates an intense electric field. A preferred pyrode may further comprise the step of ion pair alignment (polarization) in order to additively combine the small electric field contributions of myriad charge pairs into one strong electric field.

A pyrode responds to a sudden feeble warming from a pulse of coherent photons having a wavelength near the red portion of the visible light spectrum, commonly referred to as near-infrared. The human eye is known to have a transmission window in the near-infrared, at around 1120 nm. A pyrode made of pyroelectric polymer, such as PVDF, that is essentially transparent to near-infrared light, may further include an absorber to intercept and transform the pulse of near-infrared light into sudden mild warming, and ultimately into a pulsed electric field. An improved stimulator for a pyrode implanted in the eye would exploit the transmission window through the eye's focusing organs, which organs then participate as components of the stimulation system.

PVDF, for example, creates ten volts of open-circuit potential for each kelvin degree of sudden feeble warming. Only a small temperature change is needed to create a powerful electric field. When a pyrode is warmed over a minute area, such as roughly the size of a nerve cell, warming and cooling rates are most conveniently described in units of millions of degrees per second, or equivalently, in units of degrees per microsecond. Rapid warming and cooling enable pulsing the electric field at a relatively high repetition rate.

A prior art diffraction lens, comprising in part epitaxially deposited elements on a planar surface, is thin and light compared to an equivalent refracting lens. Consequently, a pair of known diffraction lenses may be closely spaced, enabling a relatively large angular deflection (scan span) using a relatively small change of lateral (in-plane) position of one diffraction lens relative to the other. Being light in weight, a diffraction lens may be positioned at video rates by a relatively small, short-stroke actuator. Prior art diffraction lenses have optical transmission efficiencies in the range of 80 to 85 percent, when scanning coherent light.

Prior art stimulators of nerve tissue (other than in the eye) rely on an implanted rechargeable electrical source, and a metallic conductor to carry current from the source to a stimulating electrode. Prior art stimulating electrodes are also made of metal. The use of metal distorts the electric field intended to be generated by the stimulating electrode. Furthermore, the use of metals invites difficulties with electrolytic polarization, corrosion, and biocompatibility. Usual prior practice stimulates several nerves in the same general area, either simultaneously, or in a prescribed sequence, which requires multiple conductors and multiple connections to the power source. In addition, metal conductors rely on bulky electrical insulation to maintain long-term function. Consequently, prior art stimulators are failure-prone, bulky, and are implanted using more invasive surgical procedures than is desirable. An improved stimulator would avoid the foregoing shortcomings by eschewing metals altogether, and instead would rely on thread-like photonic energy conduits. Furthermore, implant sites are known to dramatically increase the encapsulation thickness and reduce vascularization as a power function of the bulk of the implanted object. Therefore, an improved stimulation means would be fabricated with the least practical bulk.

Photonic conduits, also called optical fibers, or light pipes, are well known prior arts, and compared to an electrical wire, a photonic conduit may be thought of as a super-conductor, because photonic conduits deliver power sufficient for cutting, welding, and related energy-dense processes, with insignificant power losses. Known photonic conduits are made of inorganic transparent matter such as oxides, are made of organic transparent material such as polymers and elastomers, and are made of a combination of the foregoing and related materials, many of which are durable and biocompatible. The prior art of forming reliable and efficient optical couplings between photonic conduits and other elements has matured, due largely to developments by the communications industry. An improved neural stimulating means would exploit the advantageous attributes of photonic conduits.

A percutaneous lead, co-invented by the Applicant, is suited to the transmission of photonic energy from an external source, through the skin, and to an implanted stimulating means, while greatly reducing the likelihood of infection, is described in U.S. Pat. No. 3,663,965 and the referenced publication. Within the scope of U.S. Pat. No. 3,663,965 is an obvious alternative embodiment having a demountable optical coupling means incorporated in the waist of the lead.

### BRIEF SUMMARY OF THE INVENTION

A pyrode is a pyroelectric body that, when impinged by near-infrared light, creates an electrical impulse which may be used to stimulate a nerve. For partial vision restoration, the pyrode has the form of a thin polymeric lamina implanted in the retina, whereas all other portions of the apparatus, such as a scanner, reside outside the host body. Contiguous matter of the retinal pyrode enables unlimited areal addressability during scanning, and achieves higher spatial resolution than obtained by an array of discrete metal electrodes. For other than the eye, the pyrode is connected to a source of near-infrared light by a photonic conduit. The light source may be implanted as part of an electronic/photonic module, internal to which electronic components are confined, and external to which photonic energy passes by way of optical couplings and conduits. In an alternative embodiment, the electronic components and the near-infrared light source remain external to the host body, and photonic energy enters the body by way of a percutaneous lead. The percutaneous lead may have a demountable photonic coupling. All embodiments are wireless in the sense that only non-metallic, electrically non-conducting matter is used to deliver activating energy to the pyrode.

### OBJECTS OF THE INVENTION

A primary object of the invention is wireless stimulation of nerve tissue by the use of a pyrode.

Another object of the invention is to relegate all non-photonic components to locations outside the host body.

A further object of the invention is to wirelessly stimulate retinal ganglia with unlimited areal addressability

Another object of the invention is to minimize the invasiveness of implantation surgery.

Another object of the invention is to wirelessly activate a pyrode by means of a photonic conduit connected to a source of photonic energy, which source is implanted in the host body and confines non-photonic components therein, and alternatively, said source resides outside the host body and supplies photonic energy via a percutaneous lead.

Yet another object of the invention is sequentially activating multiple pyrodes from a single conduit having branches with photonic diverters, for applications such as a cochlear prosthesis, a group of motor neurons, or responsive cardiac loci.

### BRIEF DESCRIPTION OF THE DRAWINGS

FIG. 1 depicts a cross section of an eye having a pyrode implanted in its retina while being stimulated at one instant in a minute area by an external scanner.

FIG. 2 shows an oblique view of a portion of the pyrode of FIG. 1, having a focus spot, a warmed volume, a target, and an electric field.

FIG. 3 depicts a pyrode system configured to stimulate distributed neural tissue such as motor neurons using an implanted source of photonic energy.

FIG. 4 shows a more detailed cross section view of a longitudinally polarized pyrode optically coupled to and driven by a photonic conduit.

FIG. 5 shows a more detailed cross section view of a transversely polarized pyrode optically coupled to and driven by a photonic conduit.

FIG. 6 provides a partial cutaway view of pyrodes implanted in motor neurons, or in heart nerve tissue, the pyrodes activated by way of a photonic conduit through a percutaneous lead having a demountable optical coupling to an external source of photonic energy.

### DETAILED DESCRIPTION OF THE PREFERRED EMBODIMENTS

“Pyrode,” a term coined for the present invention, is an intended concatenation of “pyro” (fire) and “electrode.” Pyrode is defined herein as a body of solid, electrically non-conducting matter having a surface onto which an external source impinges a pulse of photonic energy (sudden feeble warming) that is converted internal to said body into an intense electric field manifested external to and proximate said surface.

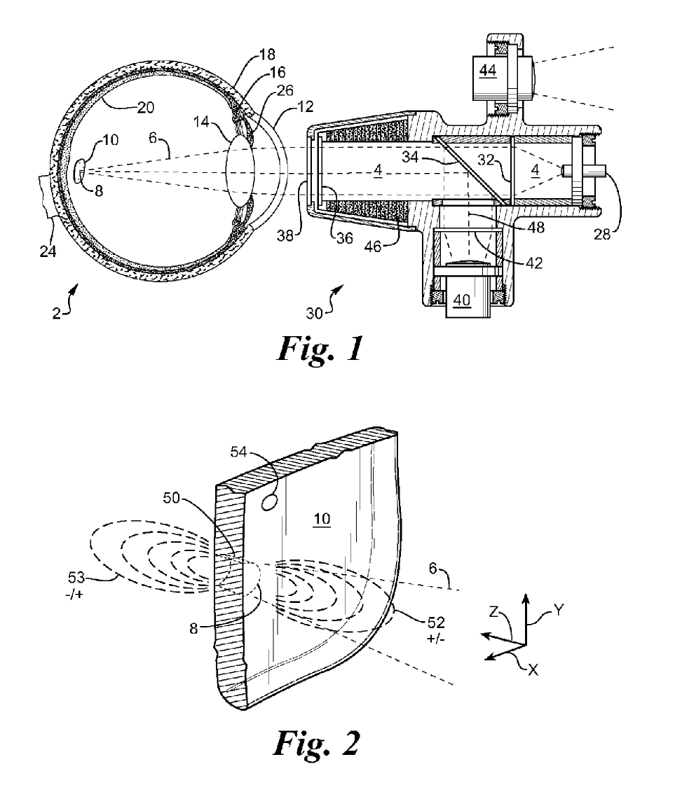

FIG. 1 depicts a cross section of a pyrode embodiment configured as a system for partial restoration of vision, consisting of scanner assembly 30 and an eye 2 having pyrode 10 implanted in retina 20. It is emphasized that this is a complete stimulating system consisting of only pyrode 10 implanted in the eye, and a scanning assembly 30 located entirely outside the eye, with no connection therebetween other than a photonic connection. Light source 28 directs near-infrared rays 4 through field lens 32, through beam splitter 34, through scanning lens 36, through imaging lens 38, and through cornea 12. The size of beam 4 is made somewhat smaller than the aperture of iris 26 in situations where a combination of dilation of iris 26, motion of eye 2, and motion of scanner 30, is allowed. Converging rays 6 are finally focused by eye lens 14 to illuminate pyrode 10 at focus spot 8 (shown at one instant of time).

Scanner assembly 30 further includes hollow, three-axis piezoelectric positioner 46 that independently positions scanning lens 36 in three directions responsive to independently controlled electrical signals in order to locate focus spot 8 anywhere in the impingeable area of pyrode 10 by adjustments in the X and Y directions (see FIG. 2), and adjustments in the Z direction to control the size of focal spot 8 and to accommodate relative movement, if there is any, between eye 2 and scanner 30.

Preferred piezoelectric positioners are described in U.S. Pat. No. 5,281,899 Electric Drive for a Segmented Transducer issued to Applicant 25 Jun. 1994, U.S. Pat. No. 5,043,621 Piezoelectric Actuator issued to Applicant 27 Aug. 1991. and U.S. Pat. No. 4,928,030 Piezoelectric Actuator issued to Applicant 22 May 1990.

Scanner assembly 30 further includes scene camera 44 that generates video signals representative of the user's field of view. Scene signals are processed by a computer (not shown) that creates activation signals for light source 28 and piezoelectric positioner 46. Scene camera 44, light source 28, the computer, and piezoelectric positioner 46 constitute the outer control loop. A second scene camera (not shown), located near the wearer's other eye, may be used to provide binocular scene image data when the apparatus is applied to both eyes. Positioning means other than a piezoelectric positioner fall within the scope of the present invention. Furthermore, it is emphasized that the pyrode is primarily responsible for enabling wireless stimulation of a retina using any scanner configured to steer and focus light, such as near-infrared light, on the pyrode.

Scanner assembly 30 further includes beam splitter 34, control camera lens 42, control camera 40, and an image processing computer (not shown), constituting the inner control loop. Processing data to calibrate and coordinate the actions of outer and inner control loops is described infra.

Scanner assembly 30 may supported to the wearer's head by a head-piece, or a frame similar to that of eyeglasses (not shown), in closely spaced relationship and essentially coaxial to eye 2.

FIG. 2 provides an oblique portion view of pyrode 10 as impinged during an instant by rays 6 originating from light source 28 and converged by eye lens 14 (FIG. 1) to focal spot 8. The impinged surface of pyrode 10 may include an antireflective coating to improve the optical efficiency. Pyrode 10 may incorporate an absorber of near-infrared light. Consequently, at the instant shown in FIG. 2, said light is absorbed in volume 50, transitorily warms the pyroelectric matter, which transitorily expands to create electric field 52 in order to stimulate proximate nerve tissue (not shown). Electric field 52 is labeled +/− to indicate that polarity may pass from positive to negative during a warming pulse. Being bipolar, electric field 53 emanating from the side opposite the illuminated side is labeled −/+. The back side of pyrode 10 may include a reflective coating to enhance optical efficiency. The absorber may have a nonlinear concentration gradient in the Z direction (FIG. 2) prescribed to render absorption of near-infrared light in a more homogeneous manner commensurate with essentially uniform sudden feeble warming of the pyroelectric material in volume 50.

Pyrode 10 further includes one or more minute targets 54. When illuminated, target 54 returns light by a combination of reflection, refraction, and diffraction to beam splitter 34, through control camera lens 42, and control camera 40 along central return ray 48. Control camera 40 and a computer (not shown) derive electrical signals representing real-time scanning characteristics, the three-space location of focus spot 8, and the location of pyrode 10 as a whole relative to scanner 30.

In the embodiment shown in FIG. 1, pyrode 10 may be implanted in the foveal region near optic nerve 24. A major object of the invention obtains since pyrode 10 has no multiconductor cable that would otherwise be implanted more invasively by way of incisions through choroid 16, sclera 18, and retina 20.

During operation, the pyrode eye stimulator uses a computer to process data from scene camera 44 in order to generate control signals for scanning positioner 46 and light source 28. A bright pixel of the user's scene will cause a focal spot 8 to stimulate a minute portion of retina 20, said portion predetermined in location by calibration. During the stimulation, light returned by a combination of reflection, refraction, and diffraction, by pyrode 10 and retina 20, pass to control camera 40 by way of beam splitter 34. A computer processes data from control camera 40, deriving signals corresponding to the size, intensity, and location of focal spot 8. Return signals are used to update the calibration of the scanning system. Return signals also convey useful information on the condition of living tissue in the vicinity of field 52.

During inner loop calibration, focal spot 8 is periodically sequentially directed at each target 54. Target locations, intensities, and other data are extracted by control camera 40 and the computer. The target portion of calibration provides sufficient data to locate pyrode 10 in three-space relative to scanner assembly 30. Target 54 is located on the impinged surface of pyrode 10, and therefore does not activate the shadowed portion of the pyrode.

During outer loop calibration, the computer expands and updates a transfer function that incorporates characteristics of the apparatus, and the user's perceptions of stimuli. Frequently, there is a displacement between a user's perceived stimulation location and the actual location of a stimulus. The calibration schemata include means to minimize activation energy applied to each portion of the pyrode commensurate with local neural sensitivity, enabling fine tuning. The transfer function is then used in part to control and operate the inner control loop.

FIG. 3 illustrates a partial cross section of a pyrode embodiment configured to activate functioning nerve tissue in portions of a host body other than the eye. This embodiment may consist of a subcutaneously implanted, hermetically sealed electronics module 62, to which is optically coupled a tree of photonic conduits terminating in at least one pyrode. Module 62 may include a monochromatic laser light source 28, a field lens 68, a window 70, and a single conduit 76 terminating in a pyrode, conduit 76 being coupled to window 70 by an optical coupling 72. Alternatively, module 62 may include a polychromatic light source 28, a field lens 68, a window 70, an energy transceiver 64, and a processor 66. Electric energy management means and inter-component wiring are omitted from the figure for clarity.

Photonic conduit 76, which is preferably an optical fiber, is optically coupled to window 70 by means of optical coupler 72, held in place by strain relief 74. Conduit 76 may terminate in a pyrode (not shown). The configurations shown by FIG. 3 provide stimulation of one nerve, or of two or more nerves in spaced apart locations.

During operation of the multi-pyrode embodiment, polychromatic light source 28 emits light 4 of a first frequency which is focused to converging rays 6 through window 70, then through optical coupler 72 into conduit 76. Diverter 78 responds to light of the first frequency in order to change the state of the diverter, in order to divert a subsequent light pulse from conduit 84 to conduit 80. The subsequent light is near-infrared which activates pyrode 82 by way of conduit 80. Similarly, diverters, conduits and pyrodes 84-102 may be individually activated by switching the state of the corresponding diverter by sending the appropriate frequency that is different from the first frequency. Preferred diverters change state by operations in the time domain, in the state of polarization, or other parameters.

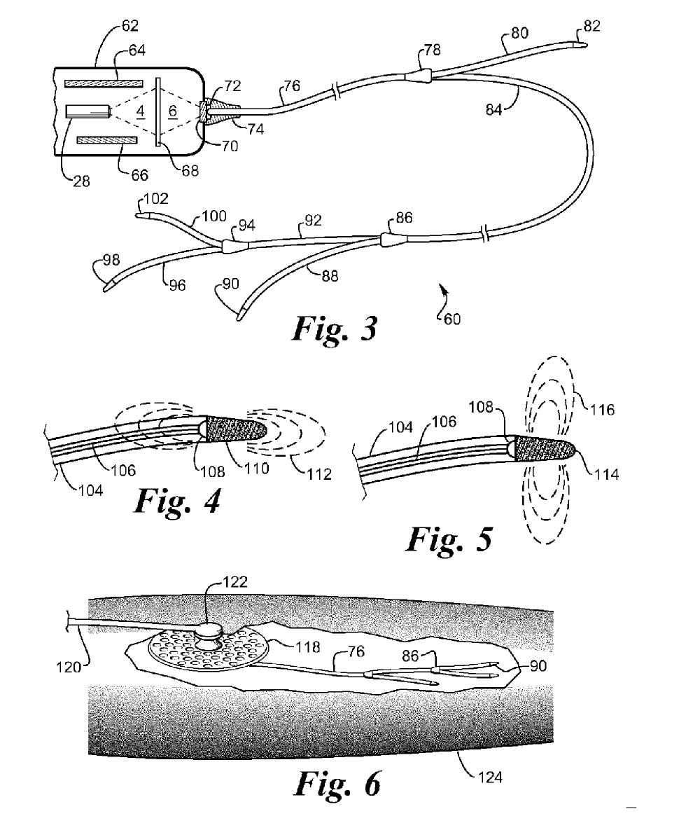

FIG. 4 shows a portion cross section view of an embodiment of a pyrode stimulator consisting of a conduit 104 containing an optical wave guide 106 which is optically coupled to pyrode 110 by optical coupler 108. Pyrode 110 is polarized during manufacture in a direction predetermined to create electric field 112 that is generally axial along the pyrode in order to stimulate nerve tissue proximate the point of pyrode 110. The point of pyrode 110 may be sharper than illustrated, and a portion of the point may lie inside a myelin sheath. A penetrating pyrode may have absorber more concentrated in its tip.

FIG. 5 shows a portion cross section of an alternative embodiment of a pyrode stimulator consisting of a conduit 104 containing an optical wave guide 106 which is optically coupled to pyrode 114 by optical coupler 108. Pyrode 114 is polarized during manufacture in a direction predetermined to create electric field 116 that is generally transverse to the axis of the pyrode. The scope of the instant invention further includes polarization in a direction angularly disposed to the pyrode axis. A pyrode that penetrates a meyelin sheath is expected to be angularly disposed to the sheath axis, wherein the tip is polarized in a direction angularly disposed to the pyrode axis in order to align the stimulating electric field along the general direction of greatest sensitivity to stimulation.

The conduits shown in the figures are exaggerated in width for drawing clarity, whereas preferred practice is very slender, thread-like conduits and pyrodes in order to minimize invasiveness during surgical implantation, and to minimize the thickness of encapsulation after long-term implantation.

An alternative embodiment of the pyrodes and conduits shown in FIG. 3 is an arrangement (not illustrated) having many pyrodes on decreasing-length branches along the distal portion of a main conduit, said branches located along a spiral that is shaped so as to be in relaxed condition when implanted in a cochlea, for the partial restoration of hearing.

FIG. 6 shows a cutaway view of an alternative embodiment having a percutaneous lead 118 to pass photonic energy from an external source (not shown) by way of external conduit 120, through demountable optical coupling 122, through skin 124, and by way of implanted conduit 76 to one or more implanted diverters such as diverter 86, and one or more pyrodes such as pyrode 90. This alternative embodiment relegates all metallic components to locations outside the body. Consequently, only simple components are implanted, and simple components are more reliable, whereas complex assemblies, such as energy management modules and lasers, are easily replaced without further surgery. A sealing cover (not shown) is installed for protection when optical coupling 122 is demounted. In similar alternative embodiments not having diverters, conduit 120, optical coupling 122, and conduit 76, may provide multiple paths for photonic energy.

Preferred percutaneous leads are described in U.S. Pat. No. 3,663,965 Bacteria-Resistant Percutaneous Device, issued to Applicant et al. 23 May 1972. A more detailed description is given in Proc. of the Conference on the Artificial Heart Program, Chapter 66, National Heart Inst., Nat. Inst. of Health, Shoreham Hotel, Washington D.C., 9-13 Jun. 1969.

An alternative embodiment of the pyrode is configured as a heart pacemaker, including either an implanted photonic energy source to activate a pyrode element by way of an optical conduit, or including a percutaneous lead that passes photonic energy from an external source, through the skin, to a pyrode element by way of an optical conduit, similar to the apparatus shown in FIG. 6. Improvements provided by pyrode pacemaking inexhaustibly include: an optical conduit that is very slender by dint of requiring no electrical insulation nor needing a coiled conductor to resist fatigue, and a pyrode element that is small enough to be “permanently” implanted inside a myelin sheath in order to reduce by orders of magnitude the energy required to initiate a heart contraction.

Obviously, many modifications and variations of the present invention are possible in light of the above teachings. It is therefore to be understood that, within the scope of the appended claims, the invention may be practiced otherwise than as specifically described.

1. A nerve stimulation device comprising,
a pyrode placed adjacent a nerve to be stimulated,
a means for impacting the pyrode with photons to create an electric field which stimulates an adjacent nerve.

2. A nerve stimulation device as in claim 1 wherein,
the pyrode contains light absorbing matter to promote sudden feeble warming.

3. A nerve stimulation device as in claim 2 wherein,
infrared light absorbing matter is used in conjunction with infrared light.

4. A nerve stimulation device as in claim 1 wherein, the pyrode has an antireflective coating.

5. A nerve stimulation device as in claim 1 wherein, the pyrode has a transmissive surface.

6. A nerve stimulation device as in claim 1 wherein,
the pyrode material is polarized to direct an electric field in a desired direction.

7. A nerve stimulation device as in claim 1 wherein,
the pyrode is adapted to be attached to a retina.

8. A nerve stimulation device as in claim 1 wherein,
a light guide is attached to the pyrode to guide photons thereto.

9. A nerve stimulation device as in claim 1 wherein,
a laser is directed at the pyrode to provide photons to a desired location.

10. A nerve stimulation device as in claim 1 wherein,
a light focusing device is directed at the pyrode to provide photons to a desired location.

11. A nerve stimulation device as in claim 1 wherein,
a light directing device is directed at the pyrode to provide photons to a desired location.

12. A nerve stimulation device as in claim 1 wherein,
the pyrode material has a shape for enhancing the electric field.

13. A nerve stimulation device as in claim 1 wherein,
a plurality of pyrodes are employed to stimulate nerve tissue at locations on the retina to enable sight.

14. A nerve stimulation device as in claim 13 wherein,
a camera and light directing means form light signals directed at selected pyrodes.

15. A nerve stimulation device as in claim 1 wherein,
a reflective location marker on the pyrode enables positioning of the light on the pyrode.

16. A method for stimulating nerves comprising attaching a pyrode adjacent a nerve, impacting the pyrode with photons to generate an electric field for stimulating the nerve.

17. A method for stimulating nerves as in claim 16 including the step of, directing the photons to a desired portion of the pyrode.

18. A method for stimulating nerves as in claim 16 including the step of, polarizing the pyrode to direct the electric field in a desired direction.

19. A method for stimulating nerves as in claim 16 including the step of, varying the intensity of the photons directed to the pyrode.

20. A method for stimulating nerves as in claim 16 including the step of, employing a light guide to direct the photons to the pyrode.
